(19) United States
(12) Patent Application Publication
(10) Pub. No.: US 2023/0134124 A1
(43) Pub. Date: May 4, 2023

(54) METHOD FOR EVALUATING THE ANTIVIRAL ABILITY OF CONVALESCENT PLASMA BY DETECTING ANTIBODY AGAINST RBD OF S PROTEIN

(71) Applicant: INSTITUTE OF BLOOD TRANSFUSION CHINESE ACADEMY OF MEDICAL SCIENCES, Sichuan (CN)

(72) Inventors: Zhong LIU, Sichuan (CN); Ling LI, Sichuan (CN); Jue WANG, Sichuan (CN)

(73) Assignee: INSTITUTE OF BLOOD TRANSFUSION CHINESE ACADEMY OF MEDICAL SCIENCES, Sichuan (CN)

(21) Appl. No.: 17/918,254

(22) PCT Filed: Apr. 24, 2020
(86) PCT No.: PCT/CN2020/086722

§ 371 Date: Oct. 11, 2022

(30) Foreign Application Priority Data
Apr. 13, 2020 (CN) 202010284126.1

(51) Int. Cl. G01N 33/569

(52) U.S. Cl. CPC G01N 33/56983; G01N 2333/165; G01N 2469/20

(57) ABSTRACT

A method for evaluating an antiviral ability of a convalescent plasma by detecting an antibody against RBD of S protein, includes: preparing a convalescent plasma; detecting the antibody against RBD of S protein according to a principle of antigen-antibody specific binding; and evaluating the antiviral ability of the convalescent plasma according to a content of the antibody against RBD in detecting the antibody against RBD of S protein.

### CROSS-REFERENCE TO RELATED APPLICATIONS

This patent application claims the benefit and priority of Chinese Patent Application No. 202010284126.1, entitled “Method for evaluating the antiviral ability of convalescent plasma by detecting antibody against RBD of S protein,” filed on Apr. 13, 2020, the disclosure of which is incorporated by reference herein in its entirety as part of the present application.

### TECHNICAL FIELD

The present disclosure relates to the technical field of biochemistry, in particular to a method for evaluating an antiviral ability of convalescent plasma by detecting an antibody against receptor-binding domain (RBD) of S protein.

### BACKGROUND

Convalescent plasma therapy is a treatment method based on plasma or plasma derivatives, that is, a method that uses plasma or plasma derivatives from patients recovered from severe infection to treat patients infected with the corresponding pathogen. The plasma of these convalescent patients contains high concentrations of specific anti-pathogen antibodies, which can neutralize pathogens after transfusion into patients, activate complement, and mediate an effective immune response, so as to achieve the purpose of treating diseases and eliminating pathogens. Convalescent plasma therapy can be traced back to the early 20th century and has been successfully applied to many infectious diseases, including anthrax, plague, scarlet fever, measles, diphtheria, dysentery, epidemic cerebrospinal meningitis, rabies, pneumococcal pneumonia, etc. During the epidemic period of severe acute respiratory syndrome (SARS) in 2003 and pandemic of H1N1 in 2009, plasma therapy also showed good results for infected patients, especially for parts of patients with ineffective drug treatment or in severe conditions.

At present, there are currently no specific drugs for targeted treatment of the emerging pathogen severe acute respiratory syndrome coronavirus 2 (SARS-CoV-2). It is going to be a while before a vaccine is developed, and the production and testing of specific antibodies requires a certain period of time. The plasma of recently cured and discharged patients contains high titers of anti-pathogen antibodies. Some studies have also pointed out that some of the new viruses isolated from the plasma of critically ill patients can be neutralized by the serum of several infected patients, indicating that there are specific neutralizing antibodies against the new virus in the serum of patients. Therefore, treatment by using convalescent plasma is expected to provide an effective means of treatment for patients infected with new pathogens, reduce mortality, and ensure the life safety of patients.

At present, the only method for evaluating the antiviral ability of convalescent plasma or immunoglobulin is neutralization test. Neutralization test has high cost, long detection period and high condition requirements, where operations have to be carried out in a P3 laboratory, and has a high safety risk due to the use of live virus.

### SUMMARY

In view of this, in order to solve the technical problems described above, an objective of the present disclosure is to develop a method for evaluating an antiviral ability of a convalescent plasma by detecting an antibody against RBD of S protein, which is simple to operate, low in cost and laboratory requirements, and high in safety, such that the detection can be performed in ordinary clinical laboratories.

Technical solutions adopted are:

A method for evaluating an antiviral ability of convalescent plasma by detecting an antibody against RBD of S protein, including the following steps:
S1. preparing a convalescent plasma;
S2. detecting the antibody against the RBD of S protein by using a principle of antigen-antibody specific binding; and
S3. evaluating the antiviral ability of convalescent plasma according to a content of the antibody against RBD in S2.

In some embodiments, in S3, when a concentration of the antibody against RBD is greater than 50-fold dilution, the convalescent plasma has a good clinically antiviral ability.

In some embodiments, in S2, the antibody against the RBD of S protein is detected by methods of enzyme-linked immunosorbent assay (ELISA) or chemiluminescence.

In some embodiments, in S1, the convalescent plasma is convalescent plasma against SARS-CoV-2 or severe acute respiratory syndrome coronavirus (SARS-CoV). Of course, the convalescent plasma includes but is not limited to this, and convalescent plasma of other pathogens is also included.

As an alternative scheme, the convalescent plasma can be replaced with immunoglobulins. That is, an alternative scheme is:
a method for evaluating an antiviral ability of an immunoglobulin by detecting an antibody against RBD of S protein, including the following steps:
S1. preparing an immunoglobulin;
S2. detecting the antibody against the RBD of S protein by using a principle of antigen-antibody specific binding; and
S3. evaluating the antiviral ability of the immunoglobulin according to a content of the antibody against RBD in S2.

Preparation of the immunoglobulin can further include concentration and purification from the convalescent plasma prepared in the alternative scheme.

An alternative scheme further included is to replace the convalescent plasma with other derivatives of the convalescent plasma.

Some beneficial effects of the embodiments in the present disclosure are as follows:

At present, the only method for evaluating the antiviral ability of convalescent plasma or immunoglobulin is neutralization test. Neutralization test has high cost, long detection period and high condition requirements, where operations have to be carried out in a P3 laboratory, and have a high safety risk due to the use of live virus. The present disclosure establishes a new method, by adopting detection targeted on the expression product of RBD, which is simple to operate, low in cost and laboratory requirements, and high in safety, and the detection can be performed in ordinary clinical laboratories.

### BRIEF DESCRIPTION OF THE DRAWINGS

FIG. 1 is a graph of experimental results showing that the method herein has a good correlation with traditional neutralization test of the live virus.

FIG. 2 is a graph of experimental results comparing the cumulative rate of no symptom improvement in the experimental group and the control group.

### DETAILED DESCRIPTION OF THE PREFERRED EMBODIMENTS

The present disclosure will be described in detail below through specific examples. However, the use and purposes of these exemplary embodiments are only used to exemplify the present disclosure, which do not constitute any form of limitation on the actual claimed scope of the present disclosure, and do not limit the claimed scope of the present disclosure.

### Example 1

A method for evaluating the antiviral ability of convalescent plasma against SARS-CoV-2 by detecting an antibody against RBD of S protein, including the following steps:
S11. preparation of the convalescent plasma (the method for preparing intravenous immunoglobulin (IVIG) was the same as the preparation of convalescent plasma, with the addition of concentration and purification steps).

200-600 mL plasma was collected using blood cell separators and closed special pipelines under a special procedure selected for plasmapheresis. The collected plasma was connected by a sterile connector, divided into 100 mL small packages, quickly placed under −40° C. for quick-freezing, and stored under −20° C.

A sample was then reserved for plasma quality testing. The interval between two plasma collections was not less than 14 days, and the plasma quality test was carried out according to the standard of GB18469.

In addition, a qualitative serological test of the SARS-CoV-2 was also carried out for reactivity study and quantitative test (titer test), with the titer not less than 50.

S2. Detection of antibody against RBD: (current methods include methods of ELISA and chemiluminescence, and this example used ELISA method).

S21. The serum containing SARS-CoV-2 was diluted with a coating solution (final concentration was 1 μg/ml-5 μg/ml), and was added into a 96-well ELISA plate with 100 μl per well for coating overnight at 4° C.;

S22. The coating solution was shaken off, then a blocking solution was added 200 μl/well for blocking overnight (or at 37° C. for 2 hours);

S23. The plate was washed thrice with washing solution, the convalescent plasma was diluted with diluent, and then the diluted convalescent plasma was added to the plate at 100 μl/well, and incubated at 37° C. for 1 hour;

S24. The plate was washed thrice with washing solution, added with an enzyme-labeled secondary antibody (anti-antibody) (diluted with diluent according to reagent instructions) at 100 μl/well, then incubated at 37° C. for 1 hour;

S25. The plate was washed three times with washing solution, add AB solution 100 μl/well, avoid light for 4 minutes to develop color at room temperature.

S26. 50 μl/well of stop solution to stop the reaction was added;

S27. OD value at 450 nm was measured by microplate reader;

S3. The concentration of the antibody against RBD was calculated. When the concentration of the antibody against RBD was greater than 50-fold dilution, it was determined that the convalescent plasma had good clinical antiviral potential.

In which, the coating solution, blocking solution, diluent, washing solution, stop solution, etc. were all conventional solutions of an ELISA method in the art. For example,

(1) coating solution (pH 9.6; 0.05 M carbonate buffer):

| | |
|---|---|
| NaCO₃ | 1.59 g, |
| NaHCO₃ | 2.93 g, |

A balance of distilled water up to 1000 ml.

(2) Washing solution (pH 7.4; PBS): 0.15 M

| | |
|---|---|
| KH₂PO₄ | 0.2 g, |
| Na₂HP0₄•12H₂0 | 2.9 g, |
| NaCl | 8.0 g, |
| KCl | 0.2 g, |
| Tween-20 | 0.5 ml(with a final concentration of 5%), |

A balance of distilled water up to 1000 ml.

(3) Diluent.

| | |
|---|---|
| bovine serum albumin(BSA) | 0.1 g, |

washing solution was added up to 100 ml;

or other serum such as sheep serum, rabbit serum mixed with the washing liquid at a ratio of 5 to 10 wt % for later use.

(4) Stop solution(2 M H₂SO₄)

distilled water 178.3 ml,

concentrated sulfuric(98 vol %) added dropwise for 21.7 ml in total.

(5) Blocking solution:

1% BSA. 1 g of bovine serum albumin (BSA) added to per 100 mL of PBST (PBST: PBS solution added with Tween-20).

### Example 2

The specific steps of this example were the same as those of Example 1. The difference was that the convalescent plasma of Example 1 was convalescent plasma against SARS-CoV-2, and in this example it was convalescent plasma against SARS-CoV.

This example was correspondingly a method for evaluating the antiviral ability of convalescent plasma against SARS-CoV by detecting an antibody against RBD of S protein.

### Example 3

This example refers to Example 1, and the difference was that IVIG was used in this example to replace the convalescent plasma of Example 1. In which, “S1. preparation of immunoglobulin,” including the following steps:
Convalescent plasma was used as raw material. Protein separation and purification by two-step ion exchange chromatography was performed, followed by nanomembrane filtration to remove virus, and intravenous human immunoglobulin preparations was prepared with glycine as stabilizer.

I. Performance of Example 1 (laboratory evaluation):

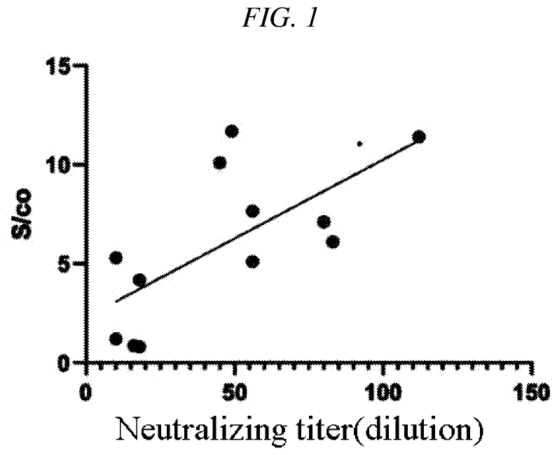

Test Method:
1. the antibody concentration in the convalescent plasma against SARS-COV-2 was detected;
2. host cells (Vero cells 10⁴) were inoculated in a 96-well plate 24 hours before infection with live SARS-CoV-2;
3. the plate was inoculated with live virus and incubated for 2 hours at 37° C., with 5 vol % CO₂, in a cell incubator;
4. the above convalescent plasma was incubated at 56° C. for 30 min, diluted 1-10 times, then added into the above cell culture plate of experimental group, and placed in an incubator containing 5 vol % CO₂ at 37° C. for 5 days, and then the cytopathic effect was observed under microscope;
5. the correlation between the concentration of antibody against RBD and the neutralization effect of live virus was analyzed, and the test results are shown in FIG. 1.

The test results showed that the method had a good correlation with the traditional live virus neutralization test, with an R value of 0.69 and a P value of 0.0139. Therefore, it was speculated that the antiviral ability of convalescent plasma could be determined by detecting the concentration of antibody against RBD.

II, Performance of Example 1 (clinical evaluation):

The concentration of antibody against RBD in convalescent plasma of blood donors was detected. The antibody concentration varied in different blood donors. When the concentration of antibody against RBD was greater than 50-fold dilution, the convalescent plasma was speculated to have a good clinical therapeutic potential.

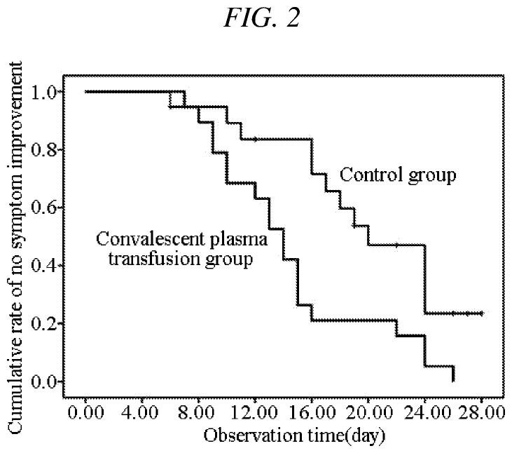

Test Method:
1. subjects were recruited and divided into an experimental group and a control group by random method;
2. IVIG or convalescent plasma with a concentration of antibody against RBD greater than 50-fold dilution was transfused into the experimental group, and other treatment methods were exactly the same as the control group.
Method of convalescent plasma transfusion was as follows:
   (1) In addition to conventional treatment, intravenous transfusion of convalescent plasma with a titer of antibody against RBD higher than 50-fold dilution was used in combination as early as possible in this method. The transfusion was conducted once on the first day. The date, the time (24-hour clock)of the beginning and end of the transfusion, as well as the volume transfused during the plasma transfusion were recorded.
   (2) Convalescent plasma transfusion principle: blood was cross-matched and transfused according to the principle of minor cross-match compatibility, plasma identified as irregular antibody negative in blood donors could be transfused according to ABO transfusion compatibility, and ABO identity plasma was preferred.
   (3) Convalescent plasma transfusion dose: the dose was determined by clinicians according to clinical conditions, patient weight and antibody titer against SARS-CoV-2. The patients in the treatment group were intravenously transfused with 100-400 mL plasma having an antibody titer higher than 50-fold dilution.
   (4) Convalescent plasma transfusion rate: the plasma was slowly transfused at a recommended rate, preferably 100mL/hour and no more than 200mL/hour, and close monitoring for transfusion adverse reactions. If adverse reactions occurred, the adverse reactions could be alleviated first by slowing down the transfusion rate. If necessary, the plasma transfusion was suspended or terminated, and the adverse reactions after plasma transfusion and the reasons for the interruption of plasma transfusion were recorded in detail.
3. The disease course of the experimental group and the control group was recorded.
4. The difference of survival status between patients transfused with the convalescent plasma having a concentration of antibody against RBD greater than 50-fold dilution and the control group was analyzed. It was found that compared with the control group, patients of the experiment group had a decreased cumulative rate of no symptom improvement, i.e., patients of the experiment group had a better cumulative rate of symptom improvement. The test results are shown in FIG. 2.

The test results showed that the cumulative rate of no symptom improvement in patients post transfusion of convalescent plasma was lower than that of the control group; i.e., the cumulative rate of symptom improvement in patients post transfusion of convalescent plasma was increased.

To summarize, in the present disclosure, the present invention adopts a new method to detect the antiviral ability of convalescent plasma or immunoglobulin by aiming at the recombinant SARS-CoV2 Spike RBD, which is responsible for SARS-CoV2 recognizing the cell surface receptor, which is simple to operate, low in cost and laboratory requirements, and high in safety, such that the operations may be performed in ordinary clinical laboratories.

The series of detailed descriptions above are only specific descriptions for feasible examples of the present disclosure, and are not intended to limit the claimed scope of the present disclosure. Those equivalent examples or modifications not departing from the technology spirit of the present disclosure should be included within the claimed scope of the present disclosure.

What is claimed is:

1. A method for evaluating an antiviral ability of a convalescent plasma by detecting an antibody against receptor binding domain (RBD) of S protein, comprising:
preparing a convalescent plasma;
detecting the antibody against RBD of S protein according to a principle of antigen-antibody specific binding; and
evaluating the antiviral ability of the convalescent plasma according to a content of the antibody against RBD in detecting the antibody against RBD of S protein.

2. The method according to claim 1, wherein in evaluating the antiviral ability of the convalescent plasma, when a concentration of the antibody against RBD is greater than 50-fold dilution, the convalescent plasma is determined to have good clinical antiviral ability.

3. The method according to claim 1, wherein in detecting the antibody against RBD of S protein, the antibody against RBD of S protein is detected by methods of enzyme-linked immunosorbent assay (ELISA) or chemiluminescence.

4. The method according to claim 1, wherein in preparing the convalescent plasma, the convalescent plasma is convalescent plasma against SARS-CoV-2 or severe acute respiratory syndrome coronavirus (SARS-CoV).

5. The method according to claim 1, wherein the convalescent plasma is replaced with an immunoglobulin.
